(19) United States
(12) Patent Application Publication
(10) Pub. No.: US 2004/0186372 A1
(43) Pub. Date: Sep. 23, 2004

(54) Mr method for the examination of a cyclically changing object

(76) Inventors: Peter Boernert, Hamburg (DE); Peter Koken, Hamburg (DE)

Correspondence Address: Thomas M Lundin, Philips Medical Systems, 595 Miner Road, Cleveland, OH, 44143, US

(21) Appl. No.: 10/297,862

(22) PCT Filed: Apr. 5, 2002
(86) PCT No.: PCT/IB02/01255

(30) Foreign Application Priority Data
Apr. 10, 2001 (DE) 101 17 787.9

(51) Int. Cl. A61B 5/055

(52) U.S. Cl. 600/410; 600/413

(57) ABSTRACT

The invention relates to an MR method for examining a cyclically changing object where a first and a second sequence act on the object during a cycle. When very many cycles are required so as to complete the second MR data set, a two-dimensional image can be reconstructed from the MR data of the first sequence so as to utilize such a two-dimensional image for monitoring purposes or as a navigator image.

The invention relates to an MR (MR=Magnetic Resonance) method for the examination of a cyclically changing object, in which method an MR sequence with parameters which are varied from one cycle to another acts on the object at the rhythm of the cycles and for a plurality of such cycles until an MR data set required for the examination has been acquired so as to be evaluated.

A method of this kind is known from an article by Stuber et al. in Radiology 1999, 212; pp. 579 to 587. In conformity with this known MR method for examinations of the heart, the MR data of, for example, ten lines in k space are acquired in each cardiac cycle, but approximately 500 acquisitions are required to reconstruct a high resolution image with, for example, 512×512×10 voxels. Also taking into account the fact that no MR data can be acquired (or the acquired data cannot be used) in phases with strong respiratory movement, it will be clear that the acquisition of such an MR data set may require, for example, approximately 15 minutes.

Because the MR data set can be completely evaluated only after this period of time has elapsed, only comparatively little information on the condition of the patient is available during the examination. The electrocardiogram required for triggering the acquisition is of limited diagnostic value only, because in MR conditions it is falsified to such an extent that essentially only the position in time of the R deflection in the electrocardiogram can be evaluated.

It is an object of the present invention to provide a method of the kind set forth such that additional information is obtained. This object is achieved by means of an MR method in accordance with the invention for the examination of a cyclically changing object, which method includes the steps of:

a) generating a first MR sequence within a part of the cycle of change of the object in order to acquire first MR data for the reconstruction of a two-dimensional or multi-dimensional MR image,

b) generating a second MR sequence within the remaining part of the cycle in order to acquire a fraction of the second MR data set required for the examination of the object,

c) repeating at least the step b) in a plurality of further cycles while varying the parameters of the second sequence in order to acquire further MR data for the second MR data set,

d) reconstructing the two-dimensional or multi-dimensional MR image during the period of time in which the step b) is repeated,

e) evaluating the second MR data set after its completion.

Thus, in accordance with the invention the first sequence is used to produce additionally a two-dimensional or multi-dimensional MR image which is reconstructed from (first) MR data which can be acquired within a part of a cycle (or a few cycles). The reconstruction of this image already takes place long before completion of the second MR data set; it can commence at least in the same cardiac cycle as that in which the first MR data was acquired. The user is thus offered the information contained therein quasi immediately instead of only after expiration of the comparatively long period of time required for the complete acquisition of the second data set.

The information contained in this fast MR image can be evaluated in various ways.

For example, in conformity with the version disclosed in claim 2 the changing object (for example, the heart) could be continuously monitored. In the version in conformity with claim 3, however, the MR image serves as a navigator image. Navigator images can be used to characterize the orientation or the position of the object being examined and to control the further examination process on the basis thereof. Until now one-dimensional “images” of the object have been generated by means of so-called navigator pulses; such images, however, are capable of characterizing the position or the shift of the object in one dimension only. The two-dimensional navigator image offers additional possibilities in this respect. However, the two-dimensional image can also be used for function studies.

Instead of forming a three-dimensional MR image from the MR data of the second sequence in conformity with claim 4, the object could also be spectroscopically examined in conformity with claim 5.

The invention can be used not only for the examination of the heart, notably of the coronary vessels, but also for the examination of other objects which are dependent on the same cycle. For example, in the case of an MR examination of the abdominal region, blood cyclically flows into and out of this region, so that the nuclear magnetization excited therein is dependent on the respective phase of a cardiac cycle in which the MR data was acquired. Thus, in this case the object does not change its position (for example, like the heart), but its properties.

The version disclosed in claim 6 offers the advantage that the second MR sequence then lies in the steadiest phase of the heart, that is, the late diastole. In that case the first MR data cannot be acquired in a similar low-motion phase, but this fact is not so important now, because in this case it suffices to acquire and reconstruct the first MR image with a lower spatial resolution, for example, 128×128 pixels.

Claim 7 describes an MR apparatus for carrying out the method in accordance with the invention and claim 8 discloses a computer program for a control unit of such an MR apparatus.

The invention will be described in detail hereinafter with reference to the drawings. Therein:

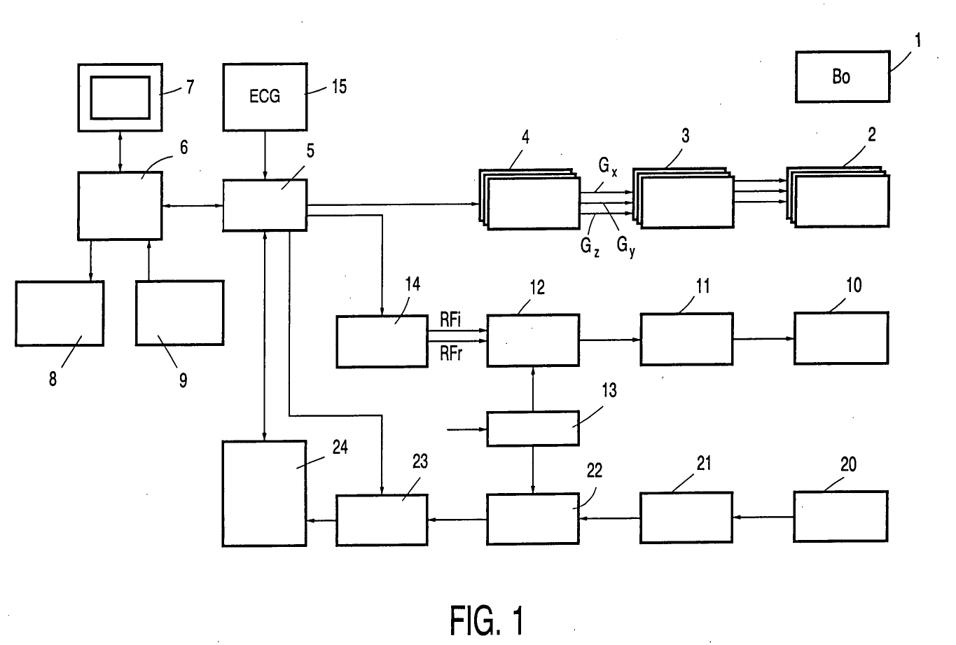

FIG. 1 shows an MR apparatus which is suitable for carrying out the invention,

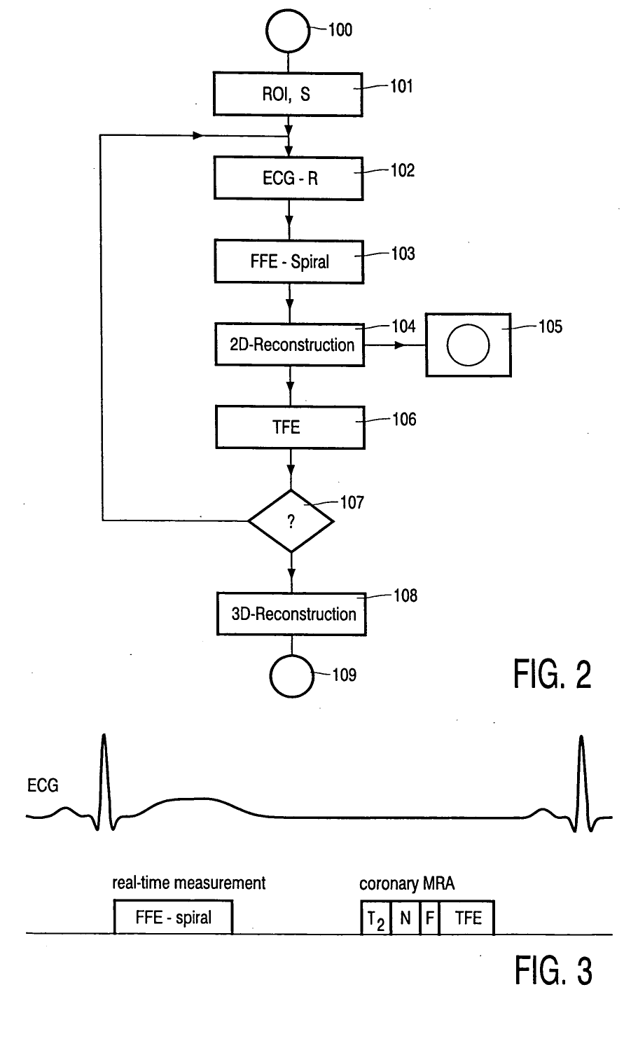

FIG. 2 shows a flow chart of the method in accordance with the invention, and

FIG. 3 shows the position of the first and the second sequence within a cycle.

The reference numeral 1 in FIG. 1 denotes a diagrammatically represented main field magnet which generates a steady and essentially homogeneous magnetic field of a strength of, for example, 1.5 Tesla in an examination zone (not shown). The direction of the magnetic field coincides with the longitudinal direction of an examination table which is not shown and on which a patient is arranged during an examination.

There is also provided a gradient coil array 2 which includes three coil systems which are suitable for generating magnetic gradient fields Gₓ, G_{y} and G_{z} which extend in the direction of the homogeneous magnetic field and have a gradient in the x direction, the y direction and the z direction, respectively. Gradient amplifiers 3 deliver the currents for the gradient coil array 2. Their variation in time is determined by a waveform generator 4, that is, for each direction separately.

The waveform generator 4 is controlled by an arithmetic and control unit 5 which calculates the variation in time of the magnetic gradient fields Gₓ, G_{y}, G_{z} as required for a given examination method and loads this variation into the waveform generator. During the MR examination these signals are fetched from the waveform generator so as to be applied to the gradient amplifiers which generate the currents required for the gradient coil array therefrom.

The control unit also co-operates with a workstation which is provided with a monitor 7 for the display of MR images. Entries can be made via a keyboard 8 or an interactive input unit 9. The control unit 5 is also connected to an electrocardiograph 15. The ECG signal delivered by the electrocardiograph 15 can be used to control an examination procedure.

The nuclear magnetization in the examination zone can be excited by RF pulses from an RF coil 10 which is connected to an RF amplifier 11 which amplifies the output signals of an RF transmitter 12. In the RF transmitter 12 the (complex) envelopes of the RF pulses are modulated with the carrier oscillations delivered by an oscillator 13, the frequency of said oscillations corresponding to the Larmor frequency (approximately 63 MHz in the case of a main magnetic field of 1.5 Tesla). The arithmetic and control unit 5 loads the complex envelope into a generator 14 which is coupled to the transmitter 12.

The MR signals generated in the examination zone are picked up by a receiving coil 20, or by a receiving coil array which consists of a plurality of receiving coils, and are amplified by an amplifier 21. In a quadrature demodulator 22 the amplified MR signal is demodulated with two 90° mutually offset carrier oscillations of the oscillator, thus generating two signals which may be considered to be the real part and the imaginary part of a complex MR signal. Discrete MR data is generated from such an MR signal by means of an analog-to-digital converter 23. Such MR data is stored in an image processing unit 24 and converted into one or more MR images by means of a suitable reconstruction method. These MR images are displayed on the monitor 7.

FIG. 2 illustrates the execution of the MR method in accordance with the invention. After the initialization (100), the user interactively selects the so-called “region of interest” (ROI) for the relevant examination in the block 101. Selection is performed on the basis of a survey image which has been formed in advance or in the step 101. In addition to the position and the dimensions of the ROI, the spatial resolution is then also specified, for example, 512×512×10 voxels. Moreover, in the same step 101 (or in a subsequent step) there is selected the position of a slice S of which MR images are to be continuously reconstructed during the examination. This slice should be situated in such a manner that it does not intersect the region of interest ROI, thus ensuring that the sequence acting on the slice does not influence the nuclear magnetization in the region of interest ROI.

The slice S may intersect, for example, the heart whereas the region of interest ROI concerns the coronary vessels which move at the rhythm of the cardiac cycle and whose nuclear magnetization changes due to blood flowing in and out. Instead of the coronary vessels themselves, other anatomical regions which change in conformity with the cardiac cycle can also be examined, for example, the abdominal region; granted, this region does not move in conformity with the cardiac cycle, but is changed by blood flowing in and out.

In the step 102 the control unit 5 evaluates the ECG signal and synchronizes the sequences subsequently generated for the region of interest ROI or the slice S, that is, in such a manner that they occupy a defined position relative to the cardiac cycle. Even though ECG signals of a patient which are acquired during an MR examination are of limited diagnostic value only, they enable reliable determination of the so-called R deflections. Thus, FIG. 3 shows the variation in time of such an ECG signal (first line) and the position in time of the subsequently generated sequences in relation to the ECG signal (second line).

In the step 103 there is first generated the sequence which is so fast that it is capable of acquiring the MR data necessary for the reconstruction of a two-dimensional MR image within a part of a cardiac cycle. An FFE spiral sequence (FFE=fast field echo) is shown by way of example; the k space is then scanned along mutually offset spiral arms so that MR data can be acquired for a low resolution MR image (for example, an image with 128×128 pixels).

This acquisition takes place in a late phase of the systole. Granted, the heart still moves in this phase, but its movement is less than the value corresponding to the spatial resolution, so that the quality of the MR image of the slice S which is subsequently reconstructed in the step 104 remains practically unaffected. The reconstruction commences in the same cardiac cycle still; it also terminates within this cardiac cycle if the image processing unit 24 is fast enough, but at least no later than after a few further cardiac cycles.

In the step 105 this image is displayed on the monitor 7. It enables, for example, the monitoring of the heart during the MR examination.

In the step 106 there is generated a second sequence which acts on the region of interest ROI. This sequence must be generated after the first sequence 103. However, this can take place simultaneously with the reconstruction of the two-dimensional MR image from the first MR data in the step 103. Because this sequence is intended to produce MR data with a high spatial resolution, it must be placed in phases of weak cardiac motion. Such a phase is the center of a diastole or the end thereof. Its distance in time from the preceding R deflection amounts to from approximately 60 to 90% of the distance between two successive R deflections.

In conformity with FIG. 3 the second sequence may first include a T₂ preparation pulse which suppresses the signal from the myocardium and from the venous blood in relation to the signal from the arterial blood. Subsequently, the sequence contains a so-called navigator pulse N which excites the nuclear magnetization in a narrow, elongate region extending perpendicularly to the diaphragm and enables measurement of the respiratory motion. Using the navigator pulse N, the MR signals acquired in given phases of the respiratory motion are suppressed or not taken into account for the reconstruction. The phase encoding can take place in the imaging part of the second sequence in dependence on the measured respiratory motion.

A fat suppression pulse F is then succeeded by the imaging part of the second sequence occurs. The latter sequence may be, for example, a so-called TFE sequence (TFE=turbo field echo) or a fast gradient echo sequence whereby, for example, the MR data of ten lines in the k space can be acquired within one cardiac cycle by means of ten successive RF pulses linked to different phase codes.

However, this constitutes only a small fraction of the MR data set required for a complete construction. Therefore, for as long as the second MR data set is not yet complete (as tested in the step 107), the steps 102 and 106 are repeated, using other phase codes of the TFE sequence, until completion of the second MR data set. Subsequently, in the step 108 a three-dimensional MR image is reconstructed from the second MR data set so as to be displayed on the monitor 7 in a suitable manner. This completes (109) the execution of the method.

The method illustrated with reference to FIG. 2 can be modified in various ways:

a) The described first sequence may be replaced by another sequence whereby the (first) MR data for the reconstruction of a low resolution two-dimensional MR image can be acquired within one cardiac cycle. Instead of a two-dimensional image, a three-dimensional MR image can also be reconstructed (while utilizing a suitably modified sequence), said three-dimensional MR image having a very low spatial resolution.

b) It is also possible to acquire the MR data for a plurality of such MR images within one cardiac cycle or to acquire only a comparatively large fraction of the data required for an MR image (having a slightly higher spatial resolution), for example, from 25 to 50%, so that a complete image of the slice can be reconstructed only from the first MR data acquired in a plurality of successive cardiac cycles.

c) On the other hand, it is not necessary to acquire MR data for a two-dimensional image in each cardiac cycle. It is not necessary either for the same slice to be imaged again and again. It may be useful for the user to modify the position of the slice interactively during the MR examination which lasts several minutes, it being essential, however, that this slice does not intersect the region of interest ROI so as to avoid interference.

d) Instead of the TFE sequence shown, any other suitable imaging sequence may be included in the second sequence. Moreover, a spectroscopic MR examination may be performed instead of a (3D) imaging examination.

e) Instead of using the two-dimensional MR image for monitoring purposes (by display on the monitor 7), the two-dimensional image may also be used as a navigator image. When this image is compared with an MR image acquired during a preceding cardiac cycle, information concerning the motion of the heart and/or its deformation or also information concerning the respiration can be extracted therefrom. Similar information is also obtained by means of the navigator pulse generated directly before the imaging part of the second sequence, but such information relates to one dimension only and not to two dimensions. The navigator pulse N which serves to measure the respiratory motions, therefore, could be dispensed with and the two-dimensional MR images could be used instead to control the phase encoding steps of the second sequence, that is, at least in successive cardiac cycles.

f) Instead of changing under the influence of the cardiac cycle, the object may also change under the influence of another cycle, for example, the respiratory cycle.

1. An MR method for the examination of a cyclically changing object, which method includes the steps of
a) generating a first MR sequence within a part of the cycle of change of the object in order to acquire first MR data for the reconstruction of a two-dimensional or multi-dimensional MR image,
b) generating a second MR sequence within the remaining part of the cycle in order to acquire a fraction of the second MR data set required for the examination of the object,
c) repeating at least the step b) in a plurality of further cycles while varying the parameters of the second sequence in order to acquire further MR data for the second MR data set,
d) reconstructing the two-dimensional or multi-dimensional MR image during the period of time in which the step b) is repeated, e) evaluating the second MR data set after its completion.

2. An MR method as claimed in claim 1, characterized in that the MR image is displayed.

3. An MR method as claimed in claim 1, characterized in that the MR image is used as a two-dimensional navigator image.

4. An MR method as claimed in claim 1, characterized in that a three-dimensional MR image is reconstructed from the second MR data set.

5. An MR method as claimed in claim 1, characterized in that an MR spectrum is derived from the second MR data set.

6. An MR method as claimed in claim 1 for the examination of the heart or the coronary vessels, the second sequence being generated and the MR signals thus generated being received each time briefly before the occurrence of the R deflection in a cardiac cycle.

7. An MR apparatus for carrying out the method claimed in claim 1, characterized in that it includes:
a magnet (1) for generating a homogeneous, steady magnetic field,
an RF transmitter (12) for generating magnetic RF pulses,
a receiver (22) for receiving MR signals,
a generator (4) for generating gradient magnetic fields with gradients exhibiting a different temporal and spatial variation,
an evaluation unit (24) for processing the MR signals received,
a device for determining the cycle of cyclical change of the object to be examined, and
a control unit (5) which controls the RF transmitter, the receiver, the generator and the evaluation unit and is programmed in such a manner that the following steps are carried out:
a) generating a first MR sequence within a part of the cycle of change of the object in order to acquire first MR data for the reconstruction of a two-dimensional MR image,
b) generating a second MR sequence within the remaining part of the cycle in order to acquire a fraction of the second MR data set required for the examination of the object,
c) repeating at least the step b) in a plurality of further cycles while varying the parameters of the second sequence in order to acquire further MR data for the second MR data set,
d) evaluating the second MR data set after its completion.

8. A computer program for a control unit for controlling an MR apparatus for carrying out the method claimed in claim 1 in such a manner that the following steps are executed:
a) generating a first MR sequence within a part of the cycle of change of the object in order to acquire first MR data for the reconstruction of a two-dimensional MR image,
b) generating a second MR sequence within the remaining part of the cycle in order to acquire a fraction of the second MR data set required for the examination of the object,
c) repeating at least the step b) in a plurality of further cycles while varying the parameters of the second sequence in order to acquire further MR data for the second MR data set,
d) evaluating the second MR data set after its completion.
